(19) United States
(12) Patent Application Publication
(10) Pub. No.: US 2020/0164121 A1
(43) Pub. Date: May 28, 2020

(54) METHOD, DEVICE AND KIT FOR THE PREPARATION OF PRP

(71) Applicant: Ahmad GHANBARI, Tehran (IR)

(72) Inventor: Ahmad GHANBARI, Tehran (IR)

(21) Appl. No.: 16/631,996

(22) PCT Filed: Jul. 18, 2017
(86) PCT No.: PCT/IB2017/054317

§ 371 Date: Jan. 17, 2020

(51) Int. Cl. A61M 1/02; A61J 1/10

(52) U.S. Cl. CPC A61M 1/029; A61M 1/0272; A61M 2039/222; A61J 1/10; A61M 2202/0427; A61M 1/0209

(57) ABSTRACT

This invention discloses a method, device and kit for the preparation of PRP. There is provided a medical device comprising a first blood bag for collection of whole blood from a patient and a second blood bag for separation of a platelet rich plasma (PRP) fraction, wherein; a whole blood inlet is provided at a first end of the first blood bag and a first blood bag outlet is provided at a second end of the first blood bag and a packed cells outlet and a plasma outlet are connected to the first blood bag outlet and a flexible plastic tube connects the plasma outlet to a plasma inlet of the third blood bag.

### TECHNICAL FIELD

The present disclosure relates to the preparation of platelet-rich plasma (PRP).

### BACKGROUND

Platelet-rich plasma (PRP) is blood plasma that has been enriched with platelets. As a concentrated source of autologous platelets, PRP contains (and releases through degranulation) several different growth factors and other cytokines that stimulate healing, e.g. of bone and soft tissue.

PRP was first developed in the 1970s and first used in Italy in 1987 in an open heart surgery procedure. PRP therapy began gaining popularity in the mid 1990s. Today, PRP injections have been safely used in many fields including wound healing, burn wound treatment, sports medicine, orthopaedics, cosmetics, faciomaxillary, gynaecology and urology.

The efficacy of certain growth factors in healing various injuries and the concentrations of these growth factors found within PRP are the theoretical basis for the use of PRP in tissue repair. The platelets collected in PRP may be activated by the addition of thrombin and calcium chloride, which induces the release of the mentioned factors from alpha granules. The growth factors and other cytokines present in PRP normally comprise: platelet-derived growth factor; transforming growth factor beta; fibroblast growth factor; insulin-like growth factor 1; insulin-like growth factor 2; vascular endothelial growth factor; epidermal growth factor; Interleukin 8; keratinocyte growth factor; and connective tissue growth factor.

Presently there are two methods of PRP preparation approved by the U.S. Food and Drug Administration. Both processes involve the collection of the patient's whole blood that is anticoagulated with citrate dextrose before undergoing two stages of centrifugation designed to separate the PRP aliquot from platelet-poor plasma (PPP) and red blood cells. In humans, the typical baseline blood platelet count is approximately 200,000 per μL. Typically, the therapeutic PRP concentrates the platelets by roughly five-fold. There is, however, broad variability in the production of PRP by various concentrating equipment and techniques.

We found some cases in patent data bases that worked on separation part of blood similar mention invention by different method and other devices, for example:

In case of WIPO data base that, Ms. Mari Giorgio disclosed a method of preparation part of blood in application No.: PCT/EP2003/010454. In mention case, they worked on some blood bag by inlet and outlet near together.

In other case that found in EPO under application No.: EP 0714667 A and WO 0174158A and WO 03063930 A, Inventers disclosed some method and devices in this technical filed.

### SUMMARY

The present inventor has identified a number of problems of the prior art PRP preparation systems.

The first problem of the prior art systems is that blood is taken/collected from the patient with a syringe. The inventor has realized that the vacuum pressure in the syringe causes damage to the platelets' sensitive membrane, which decreases the effectiveness of the resulting PRP. Therefore, it is beneficial to use a blood bag for the collection of blood instead of a syringe.

A second problem of the prior art systems are that they are “open” and thus exposed to the risk of infectious contamination. To improve sterility, it is preferred to have a closed system.

A third problem of the prior art is that a substantial portion of the platelets are damaged by contact with the hard walls of the tubes during centrifugation. This problem is avoided by the using bags instead of tubes in the centrifugation.

A fourth problem of the prior art is that high volumes of blood cannot be taken from anaemic patients or patients having large burn wounds.

The problems of the prior art has been addressed by the inventor and as a first aspect of the present disclosure, there is provided a medical device comprising a first blood bag for collection of whole blood from a patient and a second blood bag for separation of a platelet rich plasma (PRP) fraction, wherein:
a whole blood inlet is provided at a first end of the first blood bag and a first blood bag outlet is provided at a second end of the first blood bag;
a packed cells outlet and a plasma outlet are connected to the first blood bag outlet;
a first flexible plastic tube connects the plasma outlet to a plasma inlet provided at a first end of the second blood bag;
a second blood bag outlet is provided at a second end of the second bag.

Packed cells recovered from the packed cells outlet can be re injected in the blood stream of the patient and thus allows the taking of higher volumes of blood from the critically ill patient.

In one embodiment, the packed cells outlet comprises a breakable locker. Such a lock stops flow though the packed cells outlet and prevents contamination until it has been broken. When the medical device is used as intended, the breakable lock of the packed cells outlet is not broken until packed cell reinfusion is need.

When the plasma has been transferred to the second blood bag from another way there is another breakable lock.

A first branching is connected to the first blood bag outlet. In such case, the first branching comprises the packed cells outlet and the plasma outlet.

The volume of the plasma that contained platelets transferred to the second bag is typically about half of the volume of the whole blood provided in the first bag. Accordingly, the volume of the first blood bag may be higher than the volume of the second blood bag.

In one embodiment, the first flexible plastic tube comprises a breakable lock. Such a lock stops flow though the first flexible plastic tube until it has been broken.

Preferably, a platelet-rich plasma (RPR) outlet and a platelet-poor plasma (PPP) outlet are connected to the second blood bag outlet.

PPP recovered from the PPP outlet may be injected to skin for moisturizing effect, of course can be used in a moisturizing creme. Preferably, calcium and eucerin is added to the PPP for such an application.

Alternatively, PPP recovered from the PPP outlet may be used in an creme or ointment for topical treatment of a burn wound. Preferably, calcium and eucerin is added to the PPP for such an application. Accordingly, the medical device enables the provision of packed cells, PRP and PPP, which can all be used for the treatment of a patient having a burn wound. The medical device is particularly beneficial when the burn wound is large.

In one embodiment, the platelet-rich plasma (RPR) outlet comprises a breakable lock. Such a lock stops flow though the PRP outlet and prevents contamination until it has been broken. When the medical device is used as intended, the breakable lock of the RPR outlet is not broken until shortly before the PRP fraction is recovered from the second blood bag. For example, the lock in question is intended to be unbroken when the PPP fraction is transferred through the PPP outlet.

Preferably, the device further comprises a third blood bag for collection of platelet-poor plasma (PPP). In such case, a second flexible plastic tube may connect the PPP outlet to a PPP inlet provided at a first end of the third blood bag. Further, the third blood bag may comprise an outlet, for example provided at a second end of the third blood bag, for recovery of PPP from the third bag. Such an outlet may comprise a breakable lock.

In one embodiment, the second flexible plastic tube comprises a breakable lock. Such a lock stops flow though the second flexible plastic tube until it has been broken.

The medical device may comprise a second branching that is connected to the second blood bag outlet. In such case, the second branching comprises the PRP outlet and the PPP outlet.

The walls of the first blood bag, the second blood bag and (if present) the third blood bag are preferably composed of a flexible plastic material according to blood bag standards.

The first blood bag may comprise a first bag extension extending from the walls of the first blood bag. Such an extension enables fixation of the first blood bag during centrifugation. For example, holes penetrable by pins on a centrifuge may be provided in the first bag extension to allow the first blood bag to be fixed, preferably in an extended (non-creased) configuration, during centrifugation.

Likewise, the second blood bag may comprise a second bag extension extending from the walls of the second blood bag. Such an extension enables fixation of the second blood bag during centrifugation. For example, holes penetrable by pins on a centrifuge may be provided in the second bag extension to allow the second blood bag to be fixed, preferably in an extended (non-creased) configuration, during centrifugation.

To reduce the impact on the platelets and to facilitate the separation of platelets from packed cells, the second end of the first blood bag may be funnel-shaped. Alternatively, corners of the second end of the first blood bag may be rounded. However, a funnel shape is more preferred for the first blood bag.

To reduce the impact on the platelets and to facilitate the separation of platelets from (platelet poor) plasma, corners of the second end of the second blood bag may be rounded.

As a second aspect of the present disclosure, there is provided a use of a medical device according to the first aspect for separation of platelet-rich plasma (RPR) fraction from whole blood.

As a third aspect of the present disclosure, there is provided a method of preparing a platelet-rich plasma (PRP) fraction using the medical device according to the first aspect.

In this invention can be obtained PRP injected into the body by injection system or spray it on the wound by a monitoring system or can be with the help of an activator cause increase the viscosity of PRP and the produce the gel that produced gel can use in Restoration or correction Various body tissues such as bone tissue or other body tissues.

According to completely closed system and non-use of test pipe that is Conventional to prepare PRP and lack of frequent movement of blood between the syringes to tubes and conversely, it can be concluded that PRP obtained in this way and the tools are totally sterile and there is no pollution in it.

For discharging both of bags (first and second) can be used an extractor. This device is shaped like a cube that its one side has the ability to move to the other side.

with regard to this important issue that there is no hard septum surface and there are no positive and negative pressure during Bloodletting and blood injection into the PRP tool in this system and also centrifuge low RPM and with rigid sides of Second bag to Causes to no damage to the Effective platelets septum and the PRP quality will be upgraded substantially.

Shuttle form of the first bag during separating centrifuged material, Prevent mixing white blood cell phase with plasma phase containing platelets.

Input and output located both sides of the bag and in front of each other in all bags and this important issue cause's better balance during bag Centrifugation.

The method comprises the steps of:

a) subjecting the first blood bag containing whole blood to a first centrifugation to separate packed cells(RBC) from plasma that contained platelets

b) transferring a plasma that included platelets from the first blood bag through the first flexible tube to the second blood bag;

c) subjecting the second blood bag containing plasma with hanging platelets to a second centrifugation to settle platelets; and

d) removing a platelet poor plasma (PPP) fraction from the second blood bag to obtain the PRP fraction in the second blood bag.

The platelet count of the PRP fraction is typically 3-8 times higher than that of the whole blood.

Step b) may further comprise recovering of a packed cells fraction from the first blood bag after the plasma has been transferred to the second bag. At least part of such a packed cells fraction can be re injected into the bloodstream of the critical patients, preferably the patient from which the whole blood was taken.

The removal of the PPP fraction of step d) may be transferring the PPP fraction to the third blood bag. Uses of the PPP fraction are discussed above and below.

The whole blood of step a) is preferably mixed with an anticoagulant, such as CPDA1.

The first centrifugation can for example be carried out for a time period of 5-40 minutes at 500-3000 rpm. Preferably, the time period is 10-30 minutes at 750-1800 rpm.

The second centrifugation can for example be carried out for a time period of 1-10 minutes at 1500-4000 rpm. Preferably, the time period is 2-8 minutes 2000-3500 rpm.

During the first and the second centrifugation, the first and second and third blood bag, respectively, is preferably fixed in an extended configuration. In the extended configuration, the walls of the blood bag in question are not creased to any substantial degree. Pins provided on the centrifuge used for the first and second centrifugations matching holes in extensions from the walls of the blood bags may provide such a fixation.

The method may further comprise taking whole blood from a subject using a needle connected to the whole blood inlet to obtain the first blood bag containing whole blood of step a) and administration of at least part of the PRP fraction obtained in step d) to the subject. Such a method comprising the taking of whole blood and administration of PRP may be therapeutic or non-therapeutic. An example of a non-therapeutic method is a cosmetic method.

The administration method may for example be spraying, which is particularly beneficial in the treatment of large burn wounds where it is necessary to cover a large area with a limited amount of available PRP.

As a fourth aspect of the present disclosure, there is provided a kit-of-parts comprising the medical device according to the first aspect and a centrifuge. The centrifuge may be provided with pins capable of fixing a blood bag in an extended configuration during centrifugation.

### BRIEF DESCRIPTION OF THE DRAWINGS

The invention is now described, by way of example, with reference to the accompanying drawings, in which:

FIG. 1 shows an embodiment of the system/medical device of the present disclosure.

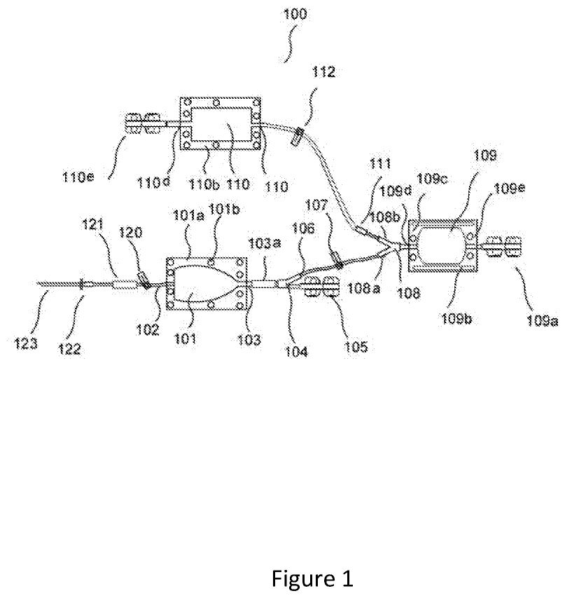

FIG. 2 shows a first blood bag of system/medical device of the present disclosure.

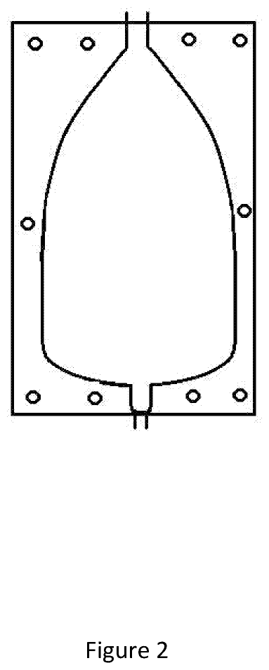

FIG. 3 shows a second blood bag of system/medical device of the present disclosure.

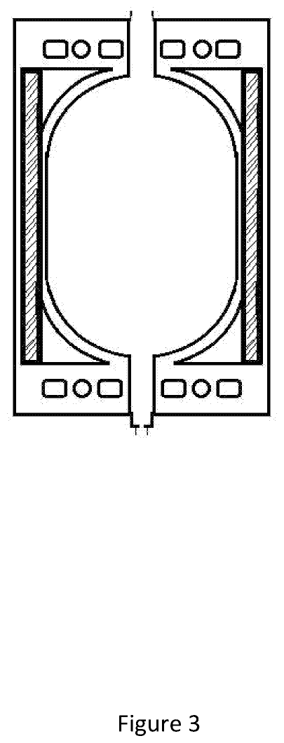

### DETAILED DESCRIPTION

Non-limiting examples of embodiments of the system and the method of the present disclosure are described below.

The system is a three-bag system 100. The three-bag system may also be referred to as a medical device.

A medical device (100) comprising a first shuttle shape blood bag (101) for collection of whole blood from a patient and a second blood bag (109) for separation of a platelet rich plasma (PRP) fraction, wherein; a needle (123) and a Luer lock (122) and a breakable locker (121) and a external clips (120) and a whole blood inlet (102) is provided at a first end (101a) of the first blood bag (101) and a first blood bag outlet (103) is provided at second end of the first blood bag (101) and a breakable locker (103a) and a packed cells outlet with safety port (105) and a plasma outlet (106) are connected to the first blood bag outlet (103) with a Three way branching (104) and breakable locker (103a) and a flexible plastic tube connects the plasma outlet (106) to a plasma inlet(108a) provided at first end (109d) of the second blood bag (109) and a second blood bag provided two hard cylindrical relatively supports (109b) on both sides that can prevent bag from falling down and bending during centrifugation, and PRP outlet (109a) is provided at second end of the second blood bag (109) and a flexible tube connect second blood bag to third blood bag (110) and third blood bag have a second connection that is outlet with safety port.

In the medical device of this invention, the first blood bag (101) is a shuttle form bag. The shuttle form first blood bag(101) have a connection for connecting the flexible plastic tube (inlet of first Blood bag) to needle (Luer lock). The shuttle shape of first blood bag (101) let us that, press the bag and move carefully the separated first phase (Plasma with Platelets) completely without mixing second phase to outlet. The shuttle shape of first blood bag (101) is a sharp in head that lead to a narrow outlet for best control on pushing the first phase of the separated plasma after first centrifuging. The shuttle shape first blood bag content adequate anticoagulant. The first three way branching (104) is connected to the first blood bag outlet (103), which first branching (104) comprises the packed cells outlet (105) and the plasma outlet (106). The volume of the first blood bag (101) is higher than the volume of the second blood bag (109). The flexible plastic tube between first blood bag and second blood bag comprises a external clips (107). The safety port (105) connected to additional port for packed cell re transfusion to patient. The platelet-rich plasma (RPR) outlet (109a) and a platelet-poor plasma (PPP) outlet (110e) have a safety port for depletion. The medical device of this invention, further comprising a third blood bag (110) for collection of platelet-poor plasma (PPP) and wherein a flexible plastic tube connects the PPP outlet (108b) from second blood bag to PPP inlet (110a) in third blood bag (110). The connecting tube between second bag to third bag that contain a internal breakable locker (111) and external clips (112). The reminded blood parts after separating of PRP can be re transfusion to critical Patients. In second bag after second centrifugation, we have two layers. For critical patinas the PPP can be move to first bag, mixing with the packed cells and re transfusion by safety port (105) or can be transfer into third bag and use for some other therapeutic purposes. In this invention, provided two hard cylindrical relatively supports (109b) on both sides that can prevent bag from falling down and bending during centrifugation. Method of preparing a platelet-rich plasma (PRP) fraction using the medical device (100) according to claims 1-16, comprising the steps of:
a) After taken blood without vacuum presser in the first blood bag
b) Subjecting the first blood bag (101) containing whole blood, by first centrifuging will be separated to packed cells and plasma that contained platelets;
c) Transferring plasma plus platelets from the first blood bag (101) through the flexible tube to the second blood bag (109).
d) After second centrifuging, there are Two fraction layers in second bag, the basal layer contains settled platelets and in the above PPP;
e) By transferring of platelet poor plasma (PPP) fraction from the second blood bag can be obtain the PRP fraction in the second blood bag (109); and
f) Before using of PRP, we shod be have a resting period for efficient expanding of settled platelets.

There is no risk of bacterial contamination because this system making totally close by connected bags. We can use a sterile spray of PRP for large damaged aria of body. There is some holes in the borders of the bags and some hooks will be enter to them for more supporting of mentions from bending and falling down due to centrifuging force. The first blood bag for carefully balancing and prevention of disturbance inlet and outlet prepare against each other. The first blood bag for prevention of mixing plasma and platelets with thrombin, inlet and outlet prepare against each other. The first blood bag for prevention of mixing plasma and platelets with RBCs of coagulated blood, inlet and outlet prepare against each other.

The walls of the first bag 101 are composed of a flexible plastic material according to blood bag standards. The first bag 101 further comprises a first bag extension 101a extending from the walls of the first bag 101. In the first bag extension 101a, holes 101b are provided to allow the first bag 101 to be fixed in an extended configuration during centrifugation. A whole blood inlet 102 is arranged a first end 101i of the first bag 101 and a blood bag outlet 103 is arranged at a second end of the first blood bag 101. To reduce the impact on the platelets and to improve the separation of platelets from packed cells, the second end of the first bag 101 is funnel-shaped. The walls of the second bag 109 are composed of a flexible plastic material according to blood bag standards.

In the method, whole blood is first collected from a patient in the first bag 101 (the blood bag). The collected volume of whole blood may for example be about 100 ml. In the first bag 101, the whole blood is mixed with an anticoagulant, preferably CPDA1. The collection and mixing with the anticoagulant are well known to the skilled person. The first bag 101 containing the whole blood mixed with the anticoagulant is then subjected to a first centrifugation, e.g. for 18 minutes at 1100 rpm, for separation of red blood cells (RBC) from plasma (containing platelets). During centrifugation, pins of the centrifuge penetrate the holes of the first bag extension to fix the first bag in an extended configuration (compaction of the bag is thus avoided). The orientation of the first bag during the first centrifugation is such that a packed cells fraction is formed at the first end of the first bag.

After the first centrifugation, the breakable locker of the first flexible tube is broke open and the first bag is gently squeezed such that the supernatant, i.e. the platelet-containing plasma, is gently pushed through the plasma outlet and the first flexible tube to the second bag. Then, the flow through the first flexible tube is stopped by the first clamp and the packed cells fraction is discharged through the packed cells outlet after that the breakable locker has been broke open. The packed cells fraction may be recovered and, for example, re injected into the blood stream if the patients is anaemic or has large burn wounds.

The concentration of platelets in the plasma transferred to the second bag is typically about 1.5 higher than in the whole blood. That means that if the platelet count in the whole blood was 200000 per ml, it is typically 340000 per ml in the plasma. If the original volume of whole blood was 100 ml, the volume of plasma transported to the second bag is typically about 50 ml (which means that about 25% of the platelets could not be separated from the packed cells if the platelet concentration 1.5 times higher in the separated plasma than in the whole blood). To further increase the platelet count, the second bag containing the plasma subjected to a second centrifugation, e.g. for 4 minutes at 2700 rpm. During the second centrifugation, the orientation of the second bag is such that platelets settle at the first end (inlet) of the second bag. During centrifugation, pins of the centrifuge penetrate the holes of the second bag extension to fix the second bag in an extended configuration. After the second centrifugation, the second bag is rested, e.g. for 30-90 minutes to allow expansion of the settled platelets.

The breakable locker of the second flexible tube is then broke open and the second bag 109 is squeezed such that the supernatant, i.e. the platelet-poor plasma (PPP), is pushed through the PPP outlet and the second flexible tube to the third bag. The PPP collected in the third bag may for example be used as a skin moisturizer.

Then, the flow through the second flexible tube is stopped by the second clamp and a plasma fraction containing the expanded platelets settled at the first end of the second bag, i.e. the platelet-rich plasma (PRP), is recovered through the PRP outlet by gently squeezing the second bag after the breakable locker of the PRP outlet has been broke open. The platelet count of the PRP fraction is typically 3-8 times higher than that of the whole blood.

In this invention can be obtained PRP injected into the body by injection system or spray it on the wound by a monitoring system or can be with the help of an activator cause increase the viscosity of PRP and the produce the gel that produced gel can use in Restoration or correction Various body tissues such as bone tissue or other body tissues.

According to completely closed system and non-use of test pipe that is Conventional to prepare PRP and lack of frequent movement of blood between the syringes to tubes and conversely, it can be concluded that PRP obtained in this way and the tools are totally sterile and there is no pollution in it.

For discharging both of bags (first and second) can be used an extractor. This device is shaped like a cube that its one side has the ability to move to the other side.

with regard to this important issue that there is no hard septum surface and there are no positive and negative pressure during Bloodletting and blood injection into the PRP tool in this system and also centrifuge low RPM and with rigid sides of Second bag to Causes to no damage to the Effective platelets septum and the PRP quality will be upgraded substantially.

Shuttle form of the first bag during separating centrifuged material, Prevent mixing white blood cell phase with plasma phase containing platelets.

Input and output located both sides of the bag and in front of each other in all bags and this important issue cause's better balance during bag Centrifugation.

The invention has mainly been described above with reference to a few embodiments. However, as is readily appreciated by a person skilled in the art, other embodiments than the ones disclosed above are equally possible within the scope of the invention, as defined by the appended patent claims.

1. A medical device, comprising:
a first blood bag for collection of whole blood from a patient and a second blood bag for separation of a platelet rich plasma (PRP) fraction, wherein a needle, a Luer lock, a breakable locker, external clips, and a whole blood inlet are provided at a first end of the first blood bag and a first blood bag outlet is provided at a second end of the first blood bag and a breakable locker;
a packed cells outlet with a safety port and a plasma outlet connected to the first blood bag outlet with a three-way branching and a breakable locker.
a flexible plastic tube connecting the plasma outlet to a plasma inlet provided at first end of the second blood bag;
the second blood bag, the second blood bag being provided with two hard cylindrical relative supports on both sides, and a PRP outlet being provided at a second end of the second blood bag; and
a flexible tube connecting the second blood bag to a third blood bag, the third blood bag having a second connection that is outlet with a safety port.

2. The medical device of claim 1, wherein the first blood bag is a shuttle form bag.

3. The medical device of claim 1, wherein the shuttle form first blood bag comprises a connection for connecting the flexible tube from an inlet of the first blood bag to the needle by way of the Luer lock.

4. The medical device of claim 1, wherein a shape of first blood bag is shuttle-shaped such that, when the first blood bag is pressed and moved, a separated first phase comprising plasma and platelets does not mix with a second phase to the outlet.

5. The medical device of claim 1, wherein a shape of first blood bag is shuttle-shaped comprising a sharp in-head leading to a narrow outlet for control on pushing a first phase of separated plasma after first centrifuging.

6. The medical device of claim 1, wherein the first blood bag is shuttled-shaped and comprises content comprising an adequate anticoagulant.

7. The medical device of claim 1, wherein the first three-way branching is connected to the first blood bag outlet, wherein the first three-way branching comprises a packed cells outlet and a plasma outlet.

8. The medical device of claim 1, wherein a capacity volume of the first blood bag is higher than a capacity volume of the second blood bag.

9. The medical device of claim 1, wherein the flexible plastic tube between first blood bag and second blood bag comprises the external clips.

10. The medical device of claim 1, wherein the safety port is connected to additional port for packed cell re transfusion to a patient.

11. The medical device of claim 1, further comprising a platelet-rich plasma (RPR) outlet and a platelet-poor plasma (PPP) outlet having a safety port for depletion.

12. The medical device of claim 1, further comprising a third blood bag for collection of platelet-poor plasma (PPP), and wherein a flexible plastic tube connects the PPP outlet from the second blood bag to a PPP inlet of the third blood bag.

13. The medical device of claim 1, wherein a connecting tube between second bag to third bag contains an internal breakable locker and external clips.

14. The medical device of claim 1, wherein remaining blood parts after a separation of PRP can be re-transfused to a patient.

15. The medical device of claim 1, wherein in second blood bag by a second centrifugation has layers and, for critical patinas, the PPP is configured to be moved to the first blood bag, mixed with the packed cells, and re-transfused by the safety port, or is configured to be transferred into the third blood bag.

16. The medical device of claim 1, wherein the two hard cylindrical relatively supports on both sides prevent the second blood bag from falling and bending during centrifugation.

17. A method of preparing a platelet-rich plasma (PRP) fraction, comprising:
providing a medical device, comprising:
a first blood bag for collection of whole blood from a patient and a second blood bag for separation of a platelet rich plasma (PRP) fraction, wherein a needle, a Luer lock, a breakable locker, external clips, and a whole blood inlet are provided at a first end of the first blood bag and a first blood bag outlet is provided at a second end of the first blood bag and a breakable locker;
a packed cells outlet with a safety port and a plasma outlet connected to the first blood bag outlet with a three-way branching and a breakable locker.
a flexible plastic tube connecting the plasma outlet to a plasma inlet provided at first end of the second blood bag;
the second blood bag, the second blood bag being provided with two hard cylindrical relative supports on both sides, and a PRP outlet being provided at a second end of the second blood bag; and
a flexible tube connecting the second blood bag to a third blood bag, the third blood bag having a second connection that is outlet with a safety port;
after taking blood without a vacuum presser in the first blood bag, subjecting the first blood bag containing whole blood to a first centrifuging force by, thereby separating packed cells and plasma containing platelets;
transferring plasma plus platelets from the first blood bag through the flexible tube to the second blood bag;
after subjecting the second blood bag to a second centrifuging force, forming two fraction layers in the second blood bag, a basal layer comprising settled platelets in the PPP;
transferring a platelet poor plasma (PPP) fraction from the second blood bag to obtain the PRP fraction in the second blood bag; and
before using the PRP fraction, providing a resting period for efficient expanding of settled platelets.

18. The method of claim 17, wherein the medical device is a closed system totally closed through connected bags.

19. The method of claim 17, further comprising applying a sterile spray of PRP for a large damaged area of body.

20. The medical device of claim 1, wherein each of the first blood bag, the second blood bag, and the third blood bag comprises holes in borders of the bags configured to receive hooks.

21-23. (canceled)
